(19) United States
(12) Patent Application Publication
(10) Pub. No.: US 2008/0161674 A1
(43) Pub. Date: Jul. 3, 2008

(54) ACTIVE IN VIVO SPECTROSCOPY

(76) Inventor: Donald Martin Monro, Beckington (GB)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/618,613

(22) Filed: Dec. 29, 2006

(51) Int. Cl. A61B 5/05

(52) U.S. Cl. 600/410

(57) ABSTRACT

Technicines for active in vivo spectroscopy are provided. An active in vivo spectroscopy technique may include transmitting a signal over a first range of frequencies to a substance, detecting a signal over a second range of frequencies from the substance in vivo, producing a frequency spectrum based on the detected signal, comparing the frequency spectrum to stored frequency spectra, and outputting a result signal based on the companson.

### FIELD

This disclosure is related to active in vivo substance spectroscopy.

### BACKGROUND

In a variety of contexts, having the ability to perform substance spectroscopy may be desirable.

### BRIEF DESCRIPTION OF THE DRAWINGS

Subject matter is particularly pointed out and distinctly claimed in the concluding portion of the specification. Claimed subject matter, however, both as to organization and method of operation, together with objects, features, and advantages thereof, may best be understood by reference of the following detailed description if read with the accompanying drawings in which:

FIG. 1 is a plot illustrating the absorption features of Herring DNA;

FIG. 2 is a plot illustrating the absorption features of Salmon DNA; and

FIG. 3 is a schematic diagram illustrating one embodiment of an apparatus for active in vivo spectroscopy.

### DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, well-known methods, procedures, components and/or circuits have not been described in detail so as not to obscure claimed subject matter.

Some portions of the detailed description which follow are presented in terms of algorithms and/or symbolic representations of operations on data bits and/or binary digital signals stored within a computing system, such as within a computer and/or computing system memory. These algorithmic descriptions and/or representations are the techniques used by those of ordinary skill in the data processing arts to convey the substance of their work to others skilled in the art. An algorithm is here, and generally, considered to be a self-consistent sequence of operations and/or similar processing leading to a desired result. The operations and/or processing may involve physical manipulations of physical quantities. Typically, although not necessarily, these quantities may take the form of electrical and/or magnetic signals capable of being stored, transferred, combined, compared and/or otherwise manipulated. It has proven convenient, at times, principally for reasons of common usage, to refer to these signals as bits, data, values, elements, symbols, characters, terms, numbers, numerals and/or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing”, “computing”, “calculating”, “determining” and/or the like refer to the actions and/or processes of a computing platform, such as a computer or a similar electronic computing device, that manipulates and/or transforms data represented as physical electronic and/or magnetic quantities and/or other physical quantities within the computing platform's processors, memories, registers, and/or other information storage, transmission, and/or display devices.

In this context, in vivo substance spectroscopy refers to methods of identifying or characterising substances in vivo by measuring data in a form known to vary between different substances so that the capability is provided to a greater or lesser extent of distinguishing between different substances for identification or other purposes. In this context, therefore, the term active in vivo substance spectroscopy includes the use of active signal emission to distinguish between or identify in vivo substances, such as for identification purposes, for example. It is noted that a variety of signals may be employed and claimed subject matter is not limited to a particular type of signal emission. To provide some examples, electromagnetic signals and/or ultrasound signals may be employed.

As is well-known, each human has a unique DNA. Despite its simple sequence of bases, the DNA molecule, in effect, codes all aspects of a particular species' characteristics. Furthermore, for each individual, it codes all the unique distinguishing biological characteristics of that individual. The DNA of an individual is also inherited at least partly from each biological parent and may be used to identify the individual or their ancestry. Work has gone on for many years, and is continuing, to relate particular DNA sequences to characteristics of a person having that DNA sequence. Thus, the DNA of an individual may reveal the genes inherited by an individual and may also, in some cases, reveal an abnormality or predisposition to certain inherited diseases, for example.

From the fields of chemistry and physics, atoms and molecules are known to provide a unique response if exposed to electromagnetic radiation, such as radio waves and/or light, for example. At the atomic or molecular level, radiation may be absorbed, reflected, or emitted by the particular atom or molecule. This produces a unique signature, although which of these phenomena take place may vary depending at least in part upon the particular frequency of the radiation impinging upon the particular atom or molecule.

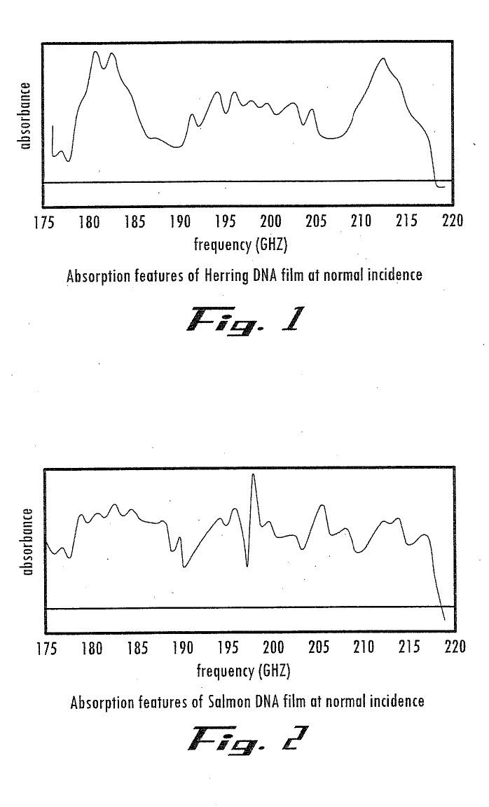

Experiments have shown that species may be distinguished by their absorption spectrum in millimetre electromagnetic waves. See Jing Ju, “Millimeter Wave Absorption Spectroscopy of Biological Polymers,” PhD Thesis, Stevens Institute of Technology, Hoboken, N.J., 2001. For example, FIGS. 1 and 2 illustrate absorption features of Herring and Salmon DNA, respectively. An approach, although claimed subject matter is not limited in scope in this respect, may include applying or observing a range of millimetre wavelengths and recording the spectral response to those millimetre wavelengths at a receiver. In such an approach, peaks and troughs in the spectral response may provide a spectrum or signature for comparison.

Therefore, waves applied to an object may be absorbed, scattered and/or reflected by the sample or the object of the radiation and the reflected, transmitted and/or scattered waves may be detected and/or recorded. At certain frequencies, modes of vibration of molecules or atoms in a sample result in radiation at that frequency being more highly absorbed, scattered or reflected compared to waves at other frequencies. At some frequencies, the sample may even emit more energy than it receives by a process that transfers energy to a resonant mode of vibration from an absorptive one.

Electromagnetic and/or mechanical resonances may be observed. In a spectral plot, this process or phenomenon may result in peaks and troughs producing a identifiable signature. In particular, in phonon resonance, molecules or portions of them may vibrate mechanically at frequencies, such as those of interest. It is well-known by the relation

v=f*l i.e. l=v/f

where v is the velocity of propagation of the wave, f is its frequency and l is the wavelength, that the wavelength is shorter for a wave that propagates more slowly. As such vibrations here are mechanical, it may be possible to induce them mechanically with sound waves of substantially the same frequency but much shorter wavelengths, e.g., ultrasound. A possible disadvantage of ultrasound is that it cannot easily be applied via free space unlike electromagnetic waves; however to induce a particular frequency of vibration the wavelength may be much shorter. This would therefore enable the use of wavelengths shorter than for electromagnetic waves.

In one embodiment, actively emitted waves in the appropriate range may be observed as absorbing and/or emitting resonances in the molecules and structures they encounter as they pass through a body to which they may be directed. For example, for such an embodiment, radiation may be applied over a broad range of frequencies, e.g., spread spectrum, and a receiver may sweep through the spectrum to determine the strength or intensity of received radiation over a suitably narrow bandwidth. In another embodiment, a receiver may instead be sensitive over a broad range of frequencies and radiation applied may be swept through the spectrum. In a third embodiment, applied and received frequencies may be swept in synchrony, although one or the other could have a narrower bandwidth to provide frequency resolution. Thus, a suitably sensitive receiver may be constructed so as to scan a suitable range of frequencies. Such a receiver may therefore detect and likewise may be employed to produce a spectrographic pattern which is characteristic of the structures and/or molecules that encountered the radiation.

Due at least in part to differences in molecular structure, different DNA and/or other substances in vivo will produce different spectrographic patterns at the receiver. Therefore, as explained in more detail below, in vivo substances, for example, may be differentiated by a signature spectrum, such as, for example, peaks and troughs in the spectrum, of actively applied radiation, such as over a suitable range of frequencies.

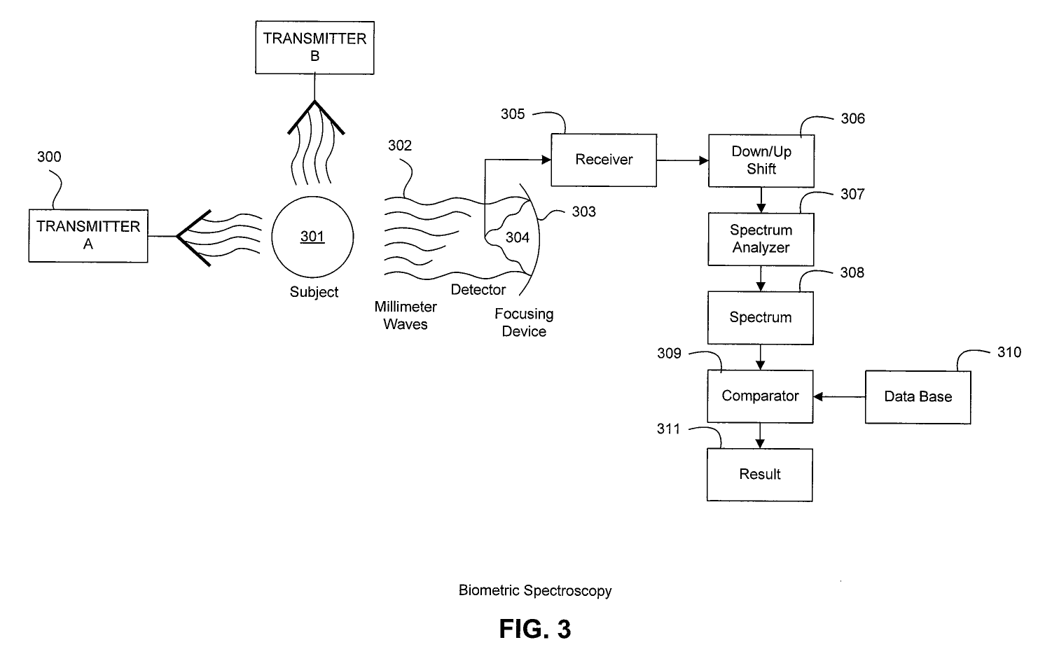

In one embodiment, for example, measurement may be accomplished via transmission. It might seem that as frequencies are swept, complex and changing patterns of reflection and scattering from internal structures, such as bone, muscle, cartilage and so on, particularly at frequencies that for some embodiments may comprise very short wavelengths, might obscure the spectrum sought. Partly this may be mitigated by choosing suitable sites for measurement, such as an earlobe, pinna or other relatively homogenous part of an anatomy. However, it is also noted that these changes should occur more slowly than peaks and troughs in the spectrum that are of interest. Thus, high pass filtering a swept receiver signal may address wide peaks and troughs due to large anatomical structures while preserving sharper peaks and troughs arising from resonances and emissions of molecules. Likewise, focusing radiation using a reflector, as shown in FIG. 3, or by some other method may be employed and may help to reduce or reject unwanted resonances by directing applied waves more precisely and/or restrict analysis to waves emanating from a desired region.

Referring to FIG. 3, for example, waves may be applied by an apparatus, such as a transmitter 300. In this particular embodiment, the waves may be modified by interaction with subject 301 to give transmitted, scattered and/or reflected waves 302 which may be focused by a focusing device 305 onto a detector 304. The reflected waves may be the result of a transmitter directing signals at an object of interest. It is noted, of course, that for this embodiment, signals are not limited to electromagnetic signals and may include other types of signals such as ultrasound and/or optical signals, for example. Signals from detector 304 may be passed to a receiver 305 which may amplify the signals before down-shifting or up-shifting the signals, at 306, to a frequency range convenient for spectrum analyzer 307. Of course, 306 may also be omitted for some embodiments. Spectrum analyzer 307 may operate in a particular frequency range, whichever may be convenient for the frequency range of interest. Resulting spectrum 308 may be compared, at 309, with previously stored spectrograms, such as, in this example, from a database 310, to produce a result 311 indicative of the quality of the match between spectrum 308 for subject 301 and spectra from database 310. Of course, this is merely one example embodiment provided for purposes of illustration. Many other embodiments are possible and are included within the scope of claimed subject matter.

It is possible that any of the frequencies mentioned above might be used and claimed subject matter is intended to cover such frequencies mentioned; however, one range to be employed, for example, may be from approximately 10 GHz to approximately 1 THz for electromagnetic signals, although, again, claimed subject matter is not limited in scope in this respect. Of course, it is difficult to predict how developments in technology may affect or influence an appropriate frequency range for use in such an application. Nonetheless, this interestingly corresponds with a prediction made by Van Zandt and Saxena in 1988, that some DNA molecules may be expected to exhibit resonances in approximately this range. See Van Zandt and Saxena, “Millimetre-microwave spectrum of DNA: Six predictions for Spectroscopy,” Phys. Rev. A 39, No. 5, pp 2692-2674, March, 1989. Likewise, a recent finding by Jing Ju indicates DNA from various species of fish and bacteria may be differentiated by millimetre wave spectroscopy in the range of approximately 180 to approximately 220 GHz. See Jing Ju, “Millimeter Wave Absorption Spectroscopy of Biological Polymers,” PhD Thesis, Stevens Institute of Technology, Hoboken, N.J., 2001.

It is noted that in some situations the emissions of interest may be of relatively low power or intensity level, so that long measurements may be desirable to obtain sufficient quanta to get a reasonable resolution of the spectrogram. In such situations, it may also be desirable to take steps to reduce measurement time. Any one of a number of techniques may be employed if this is desired. For example, one approach may be to place the individual in a suitable environment in which the background emits the radiation of a cold body. In another approach, radiation may be focused on a detector to increase its intensity, including large reflectors that at least partly or wholly surround the subject. Likewise, both approaches may be employed in some embodiments, if desired. In yet another approach, measurement time may be reduced by employing multiple receivers. For example, in one such embodiment, different receivers may be employed to cover different parts of the spectrum, such as a case in which some receivers are optical receivers and others are radio receivers, although, of course, claimed subject matter is not limited in scope in this respect. Likewise, in some embodiments, different types of signals may be used in combination, such as electromagnetic and ultrasound signals, for example.

A variety of potential applications of an embodiment, such as the one just described are possible and contemplated. However, claimed subject matter is not limited in scope to this embodiment or to these applications. Many other potential embodiments and many other applications are envisioned. Nonetheless, here we provide a few examples of illustrative applications. For example, in sports, concerted efforts are made at great expense to prevent participants from cheating by using substances that enhance performance in competition or provide an advantage if otherwise undetected. Furthermore, this may extend beyond humans, as a similar problem arises in other sporting areas, such as racing of horses, or raising dogs, to provide a few examples.

Another potential application includes medicine. Here, it would be desirable for a substance to be detected and have its concentration measured by this method. A simple non-invasive test in which an individual stands in front of a millimetre wave or infrared spectrograph and a desired substance is detected would be useful in human and veterinary medicine.

In an alternate embodiment, it may be desirable to have the capability to detect a change in a spectrograph taken on separate occasions. As mentioned previously, in such an embodiment, detecting differences between spectrographs may provide valuable for such embodiments. For example, this might be indicative of the presence of a substance in one sample, but not another, as an example. This may prove useful in many areas. In medicine, a change in biochemistry of an individual, for example, may be indicative of the appearance of a disease. By this method, it is hoped to provide the ability to screen non-invasively and potentially inexpensively for a standard range of substances, hopefully a range that is more extensive than is currently achieved through blood or urine sampling. Furthermore, after an individual, for example, has his or her characteristic biochemical spectrum registered in a system, it will be possible to monitor changes and detect a wide range of substances and conditions, even without an a priori suspected diagnosis, and without knowing what a substance is the first time it is detected.

Returning to potential implementation issues for a particular embodiment, it is noted that, in some situations, a body may not be much warmer than its surroundings, so that long measurements may be desirable to obtain sufficient quanta to get a reasonable resolution of the spectrogram. In such situations, it may also be desirable to take steps to reduce measurement time. Any one of a number of techniques may be employed if this is desired. For example, one approach may be to place the individual in a suitable environment in which the background emits the radiation of a cold body. In another approach, radiation may be focused on a detector to increase its intensity, including large reflectors that at least partly or wholly surround the subject. Likewise, both approaches may be employed in some embodiments, if desired. In yet another approach, measurement time may be reduced by employing multiple receivers. For example, in one such embodiment, different receivers may be employed to cover different parts of the spectrum, such as a case in which some receivers are optical receivers and others are radio receivers, although, of course, claimed subject matter is not limited in scope in this respect.

Likewise, a variety of spectrographic and detection techniques could be employed. In one embodiment, radio waves could be sampled and Analog-to-Digital (A/D) conversion may be employed, either directly at lower frequencies, or after modulation by a suitable carrier for down conversion to lower frequencies. In this embodiment, spectral analysis may be accomplished by applying well-known Fast Fourier Transform (FFT) techniques, for example. In such an embodiment, sampling rate and sampling duration are parameters that may affect bandwidth and line width, respectively.

In another embodiment, the frequency of the waves may be modulated upwards, for example, in one example, by an optical carrier into the optical or infra-red range and spectral analysis may be accomplished through application of standard optical spectrographic techniques, such as application of prism or prism-like technology so that light of different frequencies may be focused to detectors corresponding to a particular light frequency.

Frequencies characteristic of an individual may also be related to characteristics that differentiate the absorption or radiation characteristics of an individual, in addition to or instead of DNA resonances, depending on the particular embodiment, for example. Therefore, the range of frequencies to be employed may vary. Furthermore, claimed subject matter is not limited in scope to a particular range, of course.

It will, of course, be understood that, although particular embodiments have just been described, claimed subject matter is not limited in scope to a particular embodiment or implementation. For example, one embodiment may be in hardware, such as implemented to operate on a device or combination of devices, for example, whereas another embodiment may be in software. Likewise, an embodiment may be implemented in firmware, or as any combination of hardware, software, and/or firmware, for example. Likewise, although claimed subject matter is not limited in scope in this respect, one embodiment may comprise one or more articles, such as a storage medium or storage media. This storage media, such as, one or more CD-ROMs and/or disks, for example, may have stored thereon instructions, that if executed by a system, such as a computer system, computing platform, or other system, for example, may result in an embodiment of a method in accordance with claimed subject matter being executed, such as one of the embodiments previously described, for example. As one potential example, a computing platform may include one or more processing units or processors, one or more input/output devices, such as a display, a keyboard and/or a mouse, and/or one or more memories, such as static random access memory, dynamic random access memory, flash memory, and/or a hard drive.

In the preceding description, various aspects of claimed subject matter have been described. For purposes of explanation, specific numbers, systems and/or configurations were set forth to provide a thorough understanding of claimed subject matter. However, it should be apparent to one skilled in the art having the benefit of this disclosure that claimed subject matter may be practiced without the specific details. In other instances, well known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all such modifications and/or changes as fall within the true spirit of claimed subject matter.

1-21. (canceled)

22. A method, comprising:
(a) transmitting a signal over a first range of frequencies to a substance;
(b) detecting a signal over a second range of frequencies from the substance in vivo;
(c) producing a frequency spectrum based on the detected signal;
(d) comparing the frequency spectrum to stored frequency spectra; and
(e) outputting a result signal based on step (c).

23. The method of claim 22, wherein the detected signal is radiation transmitted in the transmitted signal modified by the substance in vivo.

24. The method of claim 22, wherein the transmitted signal is electromagnetic radiation.

25. The method of claim 22, wherein the transmitted signal is an ultrasound signal.

26. The method of claim 23, wherein the transmitted signal is optical radiation.

27. The method of claim 22, wherein the detected signal is detected for a sufficiently long period of time to a get a reasonable resolution of the frequency spectrum.

28. The method of claim 22, wherein the detected signal is detected where the substance is located in an environment in which a background of the substance emits radiation of a cold body.

29. The method of claim 22, wherein step (b) further comprises using a reflector to direct the detected signal on a detector.

30. The method of claim 22, wherein step (b) comprises using a plurality of detectors to detect the detected signal.

31. The method of claim 30, wherein respective ones of the plurality of detectors operate over different ranges of frequencies.

32. The method of claim 22, wherein at least one of the first and second range of frequencies is between about approximately 10 GHz and 1 THz.

33. The method of claim 22, wherein after step (b) and before step (c) the method further comprises:
sampling the detected signal; and
converting the sampled signal using from an analog signal to a digital signal.

34. The method of claim 33, wherein before the sampling step the method further comprises:
down converting the detected signal.

35. The method of claim 33, wherein step (c) comprises using a Fast Fourier Transform (FFT) to produce the frequency spectrum based on the detected signal.

36. The method of claim 22, wherein after step (b) and before step (c) the method further comprises:
upward modulating the second range of frequencies of the detected signal into a visible range of frequencies using an optical carrier signal.

37. The method of claim 22, wherein after step (b) and before step (c) the method further comprises:
upward modulating the second range of frequencies of the detected signal into an infrared range of frequencies using an optical carrier signal.

38. The method of claim 36, wherein step (c) comprises using an optical spectrographic system to produce the frequency spectrum based on the detected signal.

39. The method of claim 38, further comprising using prism or prism-like technology in the optical spectrographic system.

40. The method of claim 22, further comprising identifying the substance based on steps (d).

41. The method of claim 22, further comprising:
(f) transmitting a second signal over a third range of frequencies to a second substance;
(g) detecting a second signal over a fourth range of frequencies from the second substance in vivo;
(h) producing a second frequency spectrum based on the detected second signal;
(i) comparing the second frequency spectrum to stored frequency spectra; and
(j) using the first and second comparison results to distinguish between the first and second substances.

42. The method of claim 22, wherein step (b) further comprises amplifying the detected signal.

43. A system, comprising:
a transmitter configured to transmit a signal over a first range of frequencies to a substance;
a detector configured to detect a signal over a second range of frequencies from the substance in vivo;
a spectrum analyzer configured to produce a frequency spectrum based on the detected signal;
a comparator coupled to the spectrum analyzer and configured to compare the frequency spectrum to stored frequency spectra; and
a memory coupled to the comparator device and configured to store frequency spectra.

44. The system of claim 43, wherein a plurality of detectors are configured to detect the detected signal.

45. The system of claim 44, wherein respective ones of the plurality of detectors operate over different parts of the second range of frequencies.

46. The system of claim 43, further comprising:
an amplifier configured to amplify the detected signal; and
a modulator coupled to the amplifier and configured to down-shift/up-shift the amplified signal.

47. The system of claim 43, wherein the spectrum analyzer is configured to use Fast Fourier Transform (FFT).

48. A computer program product comprising a computer-useable medium having computer program logic recorded thereon, the computer program logic comprising:
(a) a computer program module that, when executed, produces a frequency spectrum based on detected signal over a range of frequencies from a substance in vivo;
(b) a computer program module that, when executed, compares the frequency spectrum to stored frequency spectra; and
(c) a computer program module that, when executed, outputs a result signal based on step (b).

49. A computer-readable medium containing instructions for controlling at least one processor by a method comprising:
(a) transmitting a signal over a first range of frequencies to a substance;
(b) detecting a sigual over a second range of frequencies from the substance in vivo;
(c) producing a frequency spectrum based on the detected signal;
(d) comparing the frequency spectrum to stored frequency spectra; and
(e) outputting a result signal based on step (c).

50. A computer-readable medium containing instructions that, when executed by a processor, causes the processor to:
(a) transmit a signal over a first range of frequencies to a substance;
(b) detect a signal over a second range of frequencies from the substance in vivo;
(c) produce a frequency spectrum based on the detected signal;
(d) compare the frequency spectrum to stored frequency spectra; and
(e) output a result signal based on step (c).
